(19) United States
(12) Patent Application Publication
(10) Pub. No.: US 2022/0133421 A1
(43) Pub. Date: May 5, 2022

(54) GUIDE WIRE CLAMPING FORCE CONTROLLING DEVICE AND METHOD FOR INTERVENTIONAL SURGICAL ROBOT

(71) Applicant: BEIJING WEMED MEDICAL EQUIPMENT CO., LTD, Beijing (CN)

(72) Inventor: Tao Huang, Beijing (CN)

(73) Assignee: BEIJING WEMED MEDICAL EQUIPMENT CO., LTD, Beijing (CN)

(21) Appl. No.: 17/229,761

(22) Filed: Apr. 13, 2021

Related U.S. Application Data
(63) Continuation of application No. PCT/CN2021/073729, filed on Jan. 26, 2021.

(30) Foreign Application Priority Data
Oct. 29, 2020 (CN) 2020111854627

(51) Int. Cl. A61B 34/37; A61B 34/00; A61B 90/00; A61B 34/30

(52) U.S. Cl. CPC A61B 34/37; A61B 34/76; A61B 2034/301; A61B 2090/064; A61B 90/06

(57) ABSTRACT

A guide wire clamping force controlling device and a method for an interventional surgical robot are disclosed. The controlling device includes a driving end, two sides of which are respectively connected with a driving part, and the two driving parts synchronously drive the driving end to move forward or backward along a direction perpendicular to the advancing direction of the vertical guide wire. The driven end includes a connecting plate, a high-precision weighing sensor, a driven end micro liner rail, a driven end slider, a driven end connecting member and a passive thread rolling part. The high-precision weighing sensor is fixed on the side of the connecting plate which is close to the guide wire, and the driven end micro liner rail is fixed on the top side.

### FIELD OF THE INVENTION

The present disclosure relates to the field of minimally invasive blood vessel technology, and more specifically, to a device and method for controlling guide wire clamping force of an interventional operation robot.

### BACKGROUND OF THE INVENTION

Minimally invasive interventional therapy for cardiovascular and cerebrovascular diseases is the main treatment for cardiovascular and cerebrovascular diseases. Compared with traditional surgery, it has obvious advantages such as small incision and short postoperative recovery time.

Cardio-cerebrovascular intervention is a process in which the doctor manually sends catheters, guide wires, and stents into the patient's body to complete the treatment.

Interventional surgery has the following two problems. First, during the operation, the DSA emits X-rays, the doctor's physical strength drop quickly, and the attention and stability will also decrease, which will cause the accuracy of the operation to decrease, and it is prone to cause improper pushing force, resulting to accidents such as vascular intimal damage, vascular perforation and rupture and endangering the lives of the patients. Second, the accumulated damage of long-term ionizing radiation will greatly increase the risk of doctors' suffering from leukemia, cancer, and acute cataracts. The phenomenon that doctors continue to accumulate radiation due to interventional surgery, which damages the professional life of doctors and restricts the development of interventional surgery, has become a problem that cannot be ignored.

The surgical method of teleoperation of catheters and guide wires with the help of robot technology can effectively cope with this problem, and can also greatly improve the accuracy and stability of the surgical operation. At the same time, it can effectively reduce the radiation damage to the interventional doctor and reduce the occurrence of intraoperative accidents. probability. Therefore, assistive robots for cardio-cerebral vascular interventional surgery have attracted more and more attention, and have gradually become the key research and development objects in the field of medical robots in today's scientific and technological powers.

In robotic surgery, the clamping of the guide wire is the basis for advancing and rotating, but the problem of over-tightening or over-loosening is likely to occur in the clamping. Over-tightening can easily lead to damage to the guide wire, and too loose can easily occur in the push Or slipping during rotation; however, there is generally no device for measuring the clamping force in the prior art, and thus the clamping force of the guide wire cannot be adjusted at any time. Therefore, how to provide a guide wire clamping force control device for an interventional surgical robot is an urgent problem for those skilled in the art.

### SUMMARY OF THE INVENTION

Therefore, the present disclosure aims to provide a guide wire clamping force controlling device of an interventional operation robot, which solves the problems that the guide wire clamping force cannot be measured and adjusted as required.

The disclosure provides a guide wire clamping force controlling device, including:

a driving end, two sides of the driving end are respectively connected with a driving part, and the two driving parts are configured to synchronously drive the driving end to move forward or backward along a direction perpendicular to an advancing direction of a guide wire; and a driven end, the driven end comprises a connecting plate, a high-precision weighing sensor, a driven end micro linear guide rail, a driven end sliding block, a driven end connecting piece and a passive thread rolling part; the high-precision weighing sensor is fixedly provided on one side of the connecting plate close to the guide wire and the driven end miniature linear guide is fixedly provided on a top side; the driven end connecting member is fixedly provided on the top of the driven end sliding block and is slidable on the driven end micro linear guide rail; the passive thread rolling part matched with the active thread rolling part of the driving end for thread rolling is fixedly provided at the top of the driven end connecting member; the high-precision weighing sensor is configured to transmit a changing signal of the received force in the thread rolling clamping process to a controlling end of the driving end of the robot propelling mechanism.

According to the above technical solution, compared with the prior art, the present disclosure discloses a guide wire clamping force controlling device for an interventional surgical robot, the driving end moves forward or backward perpendicular to a thread twisting direction of the guide wire by the driving part. The high-precision weighing sensor arranged on the connecting plate receives the change signal of the force in the thread rolling clamping process and transmits the change signal to the driving end control end of the robot propelling mechanism. The driving end control end of the robot propelling mechanism detects the change of the clamping force by comparing the value change of the feedback force, and adjusts the clamping degree of the guide wire according to the stressed condition, so that the robot adopts proper clamping force to complete the operation, and the operation can be carried out safely and reliably. Meanwhile, when the clamping force is abnormal (too much or too little), an operator can be timely reminded through the control end of the main end of the robot propelling mechanism, and the robot propelling mechanism is a safety protection device and helps a doctor to perform interventional surgery treatment better.

First, the connecting plate comprises a lower connecting plate and an upper connecting plate; the lower connecting plate comprises an integrally connected horizontal plate and a vertical plate; a first sensor fixing plate on the side close to the guide wire is provided on the top of the horizontal plate; the second sensor fixing plate arranged staggered from the first sensor fixing plate is provided on the side close to the guide wire at the bottom of the upper connecting plate; the first sensor fixing plate and the second sensor fixing plate have the same size and are both provided with a first mounting hole; and the high-precision weighing sensor is provided with a second mounting hole corresponding to the position of the first mounting hole, the first mounting hole and the second mounting hole are fixed by bolts. Thus, the high-precision weighing sensor connects the upper connecting plate and the lower connecting plate together.

Second, the passive thread rolling part comprises a fixing plate, a driven end electromagnet and a driven end active block; the fixing plate is fixedly provided at the top of the driven end connecting member, and the driven end electromagnet is vertically fixed on the fixing plate; the driven end electromagnet is magnetically connected with the driven end active block which clamps the guide wire with the driving end movable block.

Third, each driving part comprises a motor bracket, a lead screw stepping motor, a driving connecting plate, a screw nut, a driving micro liner rail and a driving slider; the bottom of the motor bracket is fixed on the shell, and the middle part of the motor bracket is used for rotatably supporting the screw stepping motor perpendicular to a twisting direction of the guide wire; an output end of the lead screw stepping motor penetrates through the driving connecting plate and is matched with the screw nut fixed on the driving connecting plate; the driving connecting plate is fixed on the side of the driving end, and the driving sliding block is arranged on the side of the driving connecting plate; the driving slider is slidable on the driving micro linear guide rail fixed on the side-wall of the shell.

The present disclosure also provides a guide wire clamping force controlling method for an interventional surgical robot, and the guide wire clamping force controlling device for an interventional surgical robot is adopted. The driving end moves forward or backward perpendicular to a thread twisting direction of the guide wire by the driving part. The high-precision weighing sensor arranged on the connecting plate receives the change signal of the force in the thread rolling clamping process and transmits the change signal to the driving end control end of the robot propelling mechanism. The driving end control end of the robot propelling mechanism detects the change of the clamping force by comparing the value change of the feedback force, and adjusts the clamping degree of the guide wire according to the stressed condition, so that the robot adopts proper clamping force to complete the operation, and the operation can be carried out safely and reliably. Meanwhile, when the clamping force is abnormal (too much or too little), an operator can be timely reminded through the control end of the main end of the robot propelling mechanism, and the robot propelling mechanism is a safety protection device and helps a doctor to perform interventional surgery treatment better.

### BRIEF DESCRIPTION OF THE DRAWINGS

In order to explain the embodiments of the present invention or the technical solutions in the prior art more clearly, the following will briefly introduce the drawings that need to be used in the description of the embodiments or the prior art. Obviously, the drawings in the following description are only embodiments of the present invention. For those of ordinary skill in the art, other drawings can be obtained according to the provided drawings without creative work.

FIG. 1 is a structural schematic diagram of the guide wire clamping force controlling device for an interventional robot by the present disclosure;

FIG. 2 is an overall schematic diagram of the driven end;

FIG. 3 is an exploded view of the driven end;

In the drawings:

100—driving end, 200—driven end, 201—connecting plate, 2011—lower connecting plate, 2012—upper connecting plate, 2013—first sensor mounting plate, 2014—second sensor mounting plate, 2015—first mounting hole, 202—high-precision weighing sensor, 2021—second mounting hole, 203—driven end micro liner rail, 204—driven end slider, 205—driven end connecting member, 206—passive thread rolling part, 2061—fixing plate, 2062—driven end electromagnet, 2063—driven end active block, 300—driving part, 301—motor bracket, 302—lead screw stepper motor, 303—driving connecting plate, 304—screw nut, 305—driving micro liner rail, 306—the driving sliding block, 400—guide wire

### DETAILED DESCRIPTION OF THE INVENTION

The embodiments of the present disclosure will be described in detail below. Examples of the embodiments are shown in the accompanying drawings, wherein the same or similar reference numerals indicate the same or similar elements or elements with the same or similar functions. The embodiments described below with reference to the drawings are exemplary, and are intended to explain the present invention, but should not be construed as limiting the present invention.

In describing the present invention, it is to be understood that the directional or positional relationships indicated by the terms “upper”, “lower”, “front”, “rear”, “left”, “right”, “vertical”, “horizontal”, “top”, “bottom”, “inner”, “outer”, and the like are based on the directional or positional relationships shown in the drawings. It is merely convenient to describe the invention and to simplify the description, rather than to indicate or imply that a device or element referred to must have a particular orientation, be constructed and operated in a particular orientation, and thus should not be construed as limiting the invention.

Furthermore, the terms “first” and “second” are used for descriptive purposes only and are not to be construed as indicating or implying relative importance or implicitly indicating the number of technical features indicated. Thus, features defining “first” and “second” may explicitly or implicitly include one or more such features. In the description of the present invention, the meaning of “a plurality” is two or more unless specifically defined otherwise.

In the present invention, the terms ‘mounted’, connected, connected, fixed”, and the like are to be construed broadly, e.g., either fixedly connected or removably connected, or integrally, unless expressly stated and defined otherwise; can be a mechanical connection or an electrical connection; they may be connected directly or indirectly through intervening media, either internal to the two elements or in an interactive relationship between the two elements. The specific meaning of the above terms in the present invention will be understood by those of ordinary skill in the art, as the case may be.

In the present invention, unless expressly stated and defined otherwise, reference to a first feature as being “above” or “below” a second feature may include reference to the first and second features being in direct contact, and reference to the first and second features not being in direct contact but being in contact by additional features therebetween. Furthermore, the first feature being “above”, “over” and “over” the second feature includes the first feature being directly above and obliquely above the second feature, or merely indicates that the first feature is at a higher level than the second feature. The first feature “below”, “below” and “beneath” the second feature includes the first feature being directly below and obliquely below the second feature, or merely indicates that the first feature has a lower horizontal height than the second feature.

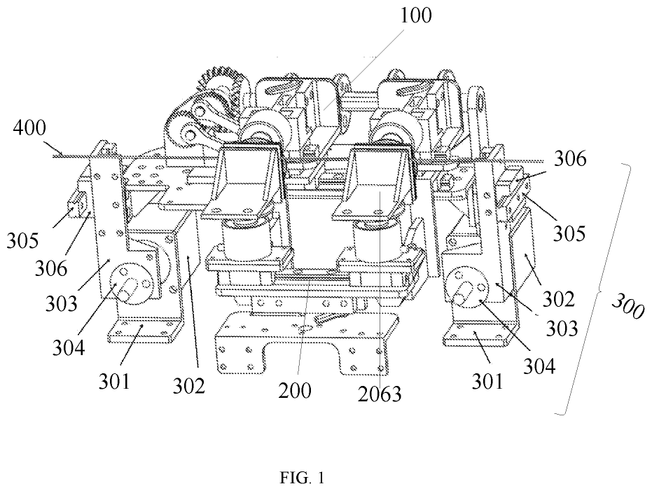

Referring to FIG. 1, the embodiment of the present disclosure provides a guide wire clamping force controlling device for an interventional surgical robot, including:

a driving end 100, two sides of the driving end are respectively connected with a driving part 300, and the two driving parts 300 are configured to synchronously drive the driving end 100 to move forward or backward along a direction perpendicular to an advancing direction of a guide wire 400; and a driven end 200, the driven end 200 comprises a connecting plate 201, a high-precision weighing sensor 202, a driven end micro linear guide rail 203, a driven end sliding block 204, a driven end connecting piece 205 and a passive thread rolling part 206; the high-precision weighing sensor 202 is fixedly provided on one side of the connecting plate 201 close to the guide wire 400 and the driven end miniature linear guide 203 is fixedly provided on a top side; the driven end connecting member 205 is fixedly provided on the top of the driven end sliding block 204 and is slidable on the driven end micro linear guide rail 203; the passive thread rolling part 206 matched with the active thread rolling part of the driving end 100 for thread rolling is fixedly provided at the top of the driven end connecting member; the high-precision weighing sensor is configured to transmit a changing signal of the received force in the thread rolling clamping process to a controlling end of the driving end of the robot propelling mechanism.

The present disclosure provides a guide wire clamping force controlling method for an interventional surgical robot, and the guide wire clamping force control device for an interventional surgical robot is adopted. The driving end control end of the robot propelling mechanism detects the change of the clamping force by comparing the value change of the feedback force, and adjusts the clamping degree of the guide wire according to the stressed condition, so that the robot adopts proper clamping force to complete the operation, and the operation can be carried out safely and reliably. Meanwhile, when the clamping force is abnormal (too much or too little), an operator can be timely reminded through the control end of the main end of the robot propelling mechanism, and the robot propelling mechanism is a safety protection device and helps a doctor to perform interventional surgery treatment better.

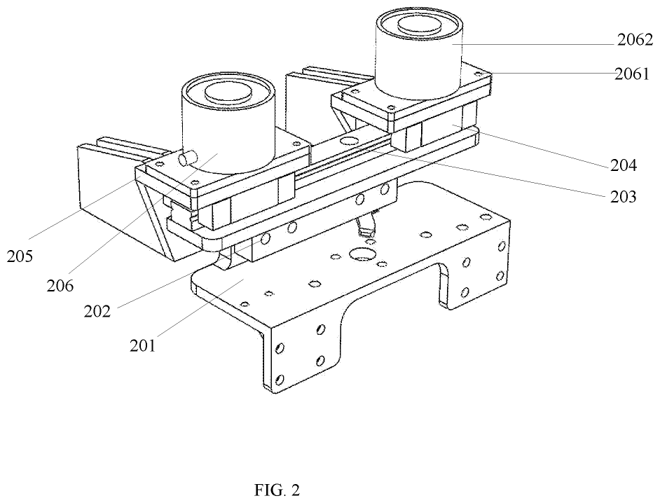

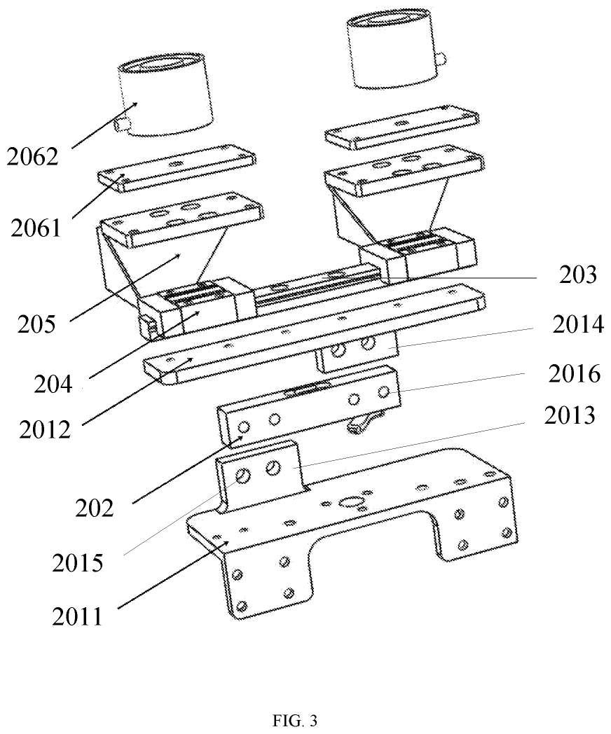

Referring to FIGS. 2 and 3, the connecting plate 201 comprises a lower connecting plate 2011 and an upper connecting plate 2012; the lower connecting plate 2011 comprises an integrally connected horizontal plate and a vertical plate; a first sensor fixing plate 2013 on the side close to the guide wire 400 is provided on the top of the horizontal plate; the second sensor fixing plate 2014 arranged staggered from the first sensor fixing plate 2013 is provided on the side close to the guide wire 400 at the bottom of the upper connecting plate 2012; the first sensor fixing plate 2013 and the second sensor fixing plate 2014 have the same size and are both provided with a first mounting hole 2015; and the high-precision weighing sensor 202 is provided with a second mounting hole 2021 corresponding to the position of the first mounting hole 2015, the first mounting hole 2015 and the second mounting hole 2021 are fixed by bolts.

In particular, the passive thread rolling part 206 comprises a fixing plate 2061, a driven end electromagnet and a driven end active block; the fixing plate 2061 is fixedly provided at the top of the driven end connecting member 205, and the driven end electromagnet 2062 is vertically fixed on the fixing plate 2061; the driven end electromagnet 2062 is magnetically connected with the driven end active block 2063 which clamps the guide wire 400 with the driving end movable block.

Advantageously, each driving part 300 comprises a motor bracket 301, a lead screw stepping motor 302, a driving connecting plate 303, a screw nut 304, a driving micro liner rail 305 and a the driving sliding block 306; the bottom of the motor bracket 301 is fixed on the shell, and the middle part of the motor bracket is used for rotatably supporting the screw stepping motor 302 perpendicular to a twisting direction of the guide wire 400; an output end of the lead screw stepping motor 302 penetrates through the driving connecting plate 303 and is matched with the screw nut 304 fixed on the driving connecting plate 303; the driving connecting plate 303 is fixed on the side of the driving end 100, and the driving sliding block 306 is arranged on the side of the driving connecting plate 303; the driving sliding block 306 is slidable on the driving micro linear guide rail 305 fixed on the side-wall of the shell.

The present disclosure also provides a guide wire clamping force controlling method for an interventional surgical robot, and the guide wire clamping force controlling device for an interventional surgical robot is adopted. The driving part drives the driving end to move forwards or backwards perpendicular to the thread twisting direction of the guide wire, during the process of thread rolling and clamping, the high-precision weighing sensor receives the force change and feeds it back to the control end of the robot propulsion mechanism, and the control end of the robot propulsion mechanism detects the clamping force by comparing the feedback force value change, and adjusts the driving part to change the clamping force according to the needs of use.

The precision of the high-precision weigh sensor is less than or equal to 0.01N weighing sensor. The high-precision weighing sensor has proper size and high sensitivity. When the moving block clamps the guide wire, a small change can be brought to the highly-precise weighing sensor in the transmission of each component. In that control end of the main end of a robot propel mechanism, the clamping force is detect by comparing the numerical value change of a high-precision weighing sensor. The two ends of the high-precision weighing sensor are respectively fixed with an upper connecting plate and a lower connecting plate, wherein the upper connection plate is provided with a driving micro linear guide rail and a secondary end electromagnet, and the lower connection plate can be fixed through the guide rails and a housing. The active end of the clamping guide wire is matched with a high-precision weight sensor under the action of a stepping screw motor, so that the clamping force of the guide wire can be controlled, that is, the motor rotates forward, The moving block attracted by the electromagnet at the active end is driven to move forwards and the movable block is close to the movable block of the driven end, so that the clamping force of the guide wire is increased. Conversely, the motor reverses rotation and the clamping force decreases.

The guide wire clamping force control device can adjust the clamping force when initialization is carried out after the guide wire is placed. The clamping force can be set by itself, and the clamping tight point or the clamping loose point can be adjusted according to actual conditions. Moreover, the change of the clamping force can be observed at any time during operation, and the clamping force can be adjusted at any time when necessary, so that the clamping device is more flexible in practical use.

Therefore, the present disclosure adopts the high-precision weighing sensor to measure the clamping force with high precision. The clamping force can be adjusted at any time through control of a lead screw stepping motor, and clinical requirements are met. The whole structure is simple, compact, stable and easy to operate. it is an important link in the whole robot.

In the description of the present specification, reference to the description of the terms “one embodiment”, “some embodiments”, “an example”, “a specific example”, or “some examples” or the like, is intended to refer to specific features, structures, materials or features that are included in at least one embodiment or example of the disclosure. In the specification, the schematic representations of the above terms are not necessarily directed to the same embodiments or examples. Moreover, the particular features, structures, materials, or features described may be combined in any one or more embodiments or examples in a suitable manner. In addition, those skilled in the art may join and combine the different embodiments or examples described in this specification.

Although the embodiments of the present disclosure have been shown and described above, it is to be understood that the embodiments described above are exemplary and not to be construed as limiting the disclosure. Variations, modifications, substitutions, and variations of the above-described embodiments may be made by one of ordinary skill in the art within the scope of the present disclosure.

What is claimed is:

1. A guide wire clamping force controlling device for an interventional surgical robot, comprising:
a driving end (100), two sides of the driving end are respectively connected with a driving part (300), and the two driving parts (300) are configured to synchronously drive the driving end (100) to move forward or backward along a direction perpendicular to an advancing direction of a guide wire (400); and
a driven end (200), the driven end (200) comprises a connecting plate (201), a high-precision weighing sensor (202), a driven end micro linear guide rail (203), a driven end sliding block (204), a driven end connecting piece (205) and a passive thread rolling part (206);
wherein the high-precision weighing sensor (202) is fixedly provided on one side of the connecting plate (201) close to the guide wire (400) and the driven end miniature linear guide (203) is fixedly provided on a top side;
the driven end connecting member (205) is fixedly provided on the top of the driven end sliding block (204) and is slidable on the driven end micro linear guide rail (203);
the passive thread rolling part (206) matched with the active thread rolling part of the driving end (100) for thread rolling is fixedly provided at the top of the driven end connecting member (205);
the high-precision weighing sensor (202) is configured to transmit a changing signal of the received force in the thread rolling clamping process to a controlling end of the driving end of the robot propelling mechanism.

2. The guide wire clamping force controlling device for an interventional surgical robot of claim 1, wherein the connecting plate (201) comprises a lower connecting plate (2011) and an upper connecting plate (2012);
the lower connecting plate (2011) comprises an integrally connected horizontal plate and a vertical plate; a first sensor fixing plate (2013) on the side close to the guide wire (400) is provided on the top of the horizontal plate;
the second sensor fixing plate (2014) arranged staggered from the first sensor fixing plate (2013) is provided on the side close to the guide wire (400) at the bottom of the upper connecting plate (2012);
the first sensor fixing plate (2013) and the second sensor fixing plate (2014) have the same size and are both provided with a first mounting hole (2015); and
the high-precision weighing sensor (202) is provided with a second mounting hole (2021) corresponding to the position of the first mounting hole (2015), the first mounting hole (2015) and the second mounting hole (2021) are fixed by bolts.

3. The guide wire clamping force controlling device for an interventional surgical robot of claim 1, wherein the passive thread rolling part (206) comprises a fixing plate (2061), a driven end electromagnet and a driven end active block; the fixing plate (2061) is fixedly provided at the top of the driven end connecting member (205), and the driven end electromagnet (2062) is vertically fixed on the fixing plate (2061); the driven end electromagnet (2062) is magnetically connected with the driven end active block (2063) which clamps the guide wire (400) with the driving end movable block.

4. The guide wire clamping force controlling device for an interventional surgical robot of claim 1, wherein each driving part (300) comprises a motor bracket (301), a lead screw stepping motor (302), a driving connecting plate (303), a screw nut (304), a driving micro liner rail (305) and a driving sliding block (306); the bottom of the motor bracket (301) is fixed on the shell, and the middle part of the motor bracket is used for rotatably supporting the screw stepping motor (302) perpendicular to a twisting direction of the guide wire (400);
an output end of the lead screw stepping motor (302) penetrates through the driving connecting plate (303) and is matched with the screw nut (304) fixed on the driving connecting plate (303);
the driving connecting plate (303) is fixed on the side of the driving end (100), and the driving sliding block (306) is arranged on the side of the driving connecting plate (303);
the driving sliding block (306) is slidable on the driving micro linear guide rail (305) fixed on the side-wall of the shell.

5. A controlling method of the guide wire clamping force device for an interventional surgical robot of claim 1, comprising:
driving, by the driving part, the driving end to move forward or backward perpendicular to a thread twisting direction of the guide wire;
receiving, by the high-precision weighing sensor, a force changing during the process of thread rolling and clamping and feeding the force changing back to a controlling end of the robot propulsion mechanism; and
detecting, by the controlling end of the robot propulsion mechanism, the clamping force by comparing the feedback force value change, and adjusting the driving part to change a clamping force accordingly.
